(19) United States
(12) Patent Application Publication
(10) Pub. No.: US 2017/0119445 A1
(43) Pub. Date: May 4, 2017

(54) BONE SCREW INCLUDING A DUAL THREAD CLOSURE MEMBER

(71) Applicant: Zimmer Spine, Inc., Edina, MN (US)

(72) Inventor: Jeremy J. Lemoine, Leander, TX (US)

(21) Appl. No.: 15/397,959

(22) Filed: Jan. 4, 2017

Related U.S. Application Data
(63) Continuation of application No. 13/771,442, filed on Feb. 20, 2013, now abandoned.
(60) Provisional application No. 61/601,809, filed on Feb. 22, 2012.

(51) Int. Cl. A61B 17/86; A61B 17/70

(52) U.S. Cl. CPC A61B 17/8605; A61B 17/7032; A61B 17/863

(57) ABSTRACT

A bone screw including a housing including a threaded opening defined between first and second legs extending from a base portion of the housing, and a threaded closure member configured to be threaded into the threaded opening of the housing between the first and second legs to secure a connecting member in a channel of the housing. The threaded closure member includes a first thread and a second thread intertwined with the first thread, and the threaded opening includes a first discontinuous thread and a second discontinuous thread intertwined with the first discontinuous thread.

### CROSS-REFERENCE TO RELATED APPLICATIONS

This application claims the benefit of U.S. Provisional Application No. 61/601,809, filed on Feb. 22, 2012, the entire disclosure of which is incorporated herein by reference.

### TECHNICAL FIELD

The disclosure is directed to a vertebral anchor including a threaded closure member having a dual threaded portion. More particularly, the disclosure is directed to a bone screw including a threaded closure member having first and second intertwined threads threadably engageable with a threaded opening of the housing of the bone screw having first and second intertwined discontinuous threads.

### BACKGROUND

The spinal column of a patient includes a plurality of vertebrae linked to one another by facet joints and an intervertebral disc located between adjacent vertebrae. The facet joints and intervertebral disc allow one vertebra to move relative to an adjacent vertebra, providing the spinal column a range of motion. Diseased, degenerated, damaged, or otherwise impaired facet joints and/or intervertebral discs may cause the patient to experience pain or discomfort and/or loss of motion, thus prompting surgery to alleviate the pain and/or restore motion of the spinal column.

One possible method of treating these conditions is to immobilize a portion of the spine to allow treatment. Traditionally, immobilization has been accomplished by rigid or dynamic stabilization. For example, in a conventional spinal fusion procedure, a surgeon restores the alignment of the spine or the disc space between vertebrae by installing a rigid or dynamic fixation rod or spacer between pedicle screws, or other vertebral anchors secured to adjacent vertebrae. Bone graft is placed between the vertebrae or along the posterior spinal elements, and the fixation rod or spacer cooperates with the screws to immobilize the two vertebrae relative to each other so that the bone graft may fuse with the vertebrae.

Accordingly, it is desirable to provide alternative designs and constructions of vertebral anchors, such as bone screws, for securing stabilization systems to a vertebral segment of a spinal column during a medical procedure.

### SUMMARY

The disclosure is directed to several alternative designs, materials and methods of manufacturing medical device structures and assemblies, and uses thereof.

Accordingly, one illustrative embodiment is a vertebral fastener including a housing with a bone engaging portion extending therefrom, and a closure member configured to threadably engage the housing. The housing includes a first leg and a second leg extending from a base portion of the housing and defining a channel between the first leg and the second leg for receiving a connecting member therethrough. The closure member is configured to threadably engage the first and second legs of the housing. The closure member includes a first thread and a second thread intertwined with the first thread. The first thread has a start proximate a lower surface of the closure member and the second thread has a start proximate the lower surface of the closure member.

Another illustrative embodiment is a bone screw including a housing, a threaded shaft, and a threaded closure member. The housing includes a base portion and first and second legs extending from the base portion. The first and second legs define a threaded opening extending into the housing from an upper extent of the housing. The housing also includes a channel intersecting the threaded opening for receiving a connecting member therethrough. The threaded shaft extends from the housing for threadably engaging a bony structure. The threaded closure member is configured to be threaded into the threaded opening of the housing between the first and second legs to secure a connecting member in the channel. The threaded closure member includes a first thread and a second thread intertwined with the first thread, and the threaded opening includes a first discontinuous thread and a second discontinuous thread intertwined with the first discontinuous thread.

Yet another illustrative embodiment is a method of securing a connecting member within a channel of a vertebral fastener. The method includes inserting a connecting member within a channel of a housing of a vertebral fastener between a first leg and a second leg of the housing. A closure member is then threadably engaged within a threaded opening of the housing between the first leg and the second leg of the housing. The closure member includes a first thread and a second thread intertwined with the first thread. The first thread has a start proximate a lower surface of the closure member and the second thread has a start proximate the lower surface of the closure member. In some instances, the start of the first thread of the closure member threadably initiates threaded. engagement with the first leg of the housing substantially simultaneously as the start of the second thread initiates threaded engagement with the second leg of the housing.

The above summary of some example embodiments is not intended to describe each disclosed embodiment or every implementation of the aspects of the disclosure.

### BRIEF DESCRIPTION OF THE DRAWINGS

The aspects of the disclosure may be more completely understood in consideration of the following detailed description of various embodiments in connection with the accompanying drawings, in which:

FIG. 1 is a perspective view of an exemplary vertebral anchor, shown as a bone screw;

FIG. 2 is a cross-sectional view of the bone screw of FIG. 1;

FIGS. 3 and 4 are perspective views of an exemplary closure member of the vertebral anchor of FIG. 1;

FIG. 5 is a bottom view of the closure member of the vertebral anchor of FIG. 1;

FIGS. 6A and 6B are perspective views of an exemplary housing of the vertebral anchor of FIG. 1;

FIG. 7 is a top view of the housing of the vertebral anchor of FIG. I; and

FIG. 8 is a cross-sectional view of the closure member and the housing of the vertebral anchor of FIG. 1.

While the aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein, the term “thread” refers to a projecting rib extending helically along a member.

As used herein, the term “external thread” refers to a thread extending radially outward on an outside of a member.

As used herein, the term “internal thread” refers to a thread extending radially inward on an inside of a member.

As used herein, the term “major diameter” refers to the largest diameter of an external or internal thread.

As used herein, the term “minor diameter” refers to the smallest diameter of an external or internal thread.

As used herein, the term “crest” refers to the surface of a thread corresponding to the major diameter of an external thread or the minor diameter of an internal thread.

As used herein, the term “root” refers to the surface of a thread corresponding to the minor diameter of an external thread or the major diameter of an internal thread.

As used herein the term “flank” refers to a surface of a thread connecting the crest and the root.

As used herein, the term “thread start” or “start” refers to the point where a thread begins.

As used herein, the term “lead” refers to the axial distance between adjacent crests of a thread during one complete revolution (360°) of the thread.

As used herein, the term “pitch” refers to the axial distance between adjacent crests along a thread portion of a member. In a threaded member including a single helical thread, the lead and the pitch are equal. In a threaded member including multiple intertwined threads, the lead is equal to the pitch multiplied by the number of threads.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

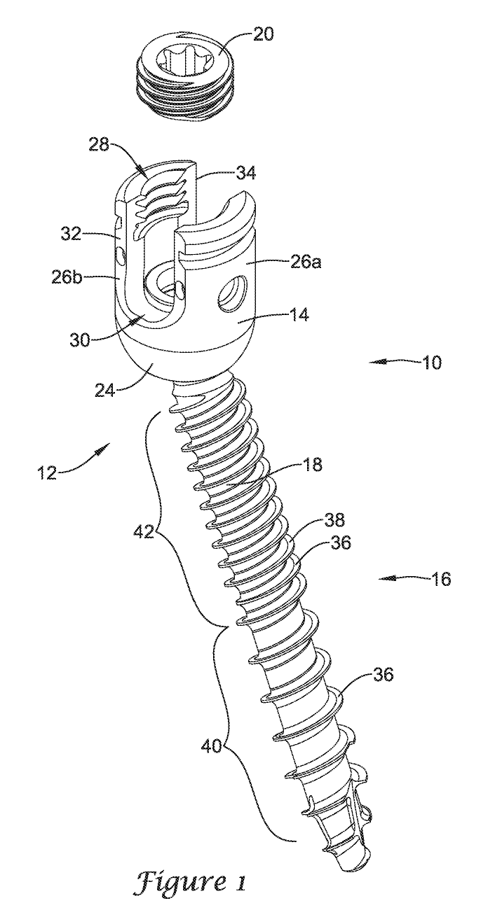

An exemplary vertebral anchor 10 is illustrated at FIG. 1. Although the vertebral anchor 10 is depicted as a threaded vertebral fastener (e.g., pedicle screw, bone screw), in some embodiments the vertebral anchor 10 may be a vertebral hook (e.g., laminar hook) or another type of fastening member for attachment to a bony structure such as a vertebra of the spinal column.

The vertebral anchor 10, shown as a bone screw 12, may include a housing 14 and a bone engagement portion 16 extending from the housing 14. In some embodiments, the bone engagement portion 16 may be a shaft 18 of the bone screw 12 extending from the housing 14 along a longitudinal axis of the bone screw 12. In embodiments in which the vertebral anchor 10 is a vertebral hook, the bone engagement portion 16 could be configured as a hook to be secured to a bony structure, for example. In some embodiments, the bone screw 12 may be a monoaxial screw in which the housing 14 is stationary relative to the shaft 18, and in other embodiments the bone screw 12 may be a polyaxial screw in which the housing 14 is actuatable (e.g., pivotable, rotatable) relative to the shaft 18. In some embodiments, the shaft 18 may be configured to be installed into a bony region of a vertebra of the spinal column. For example, the shaft 18 may be installed into a pedicle of a vertebra, or other region of a vertebra.

In some embodiments, the shaft 18 may be a threaded region having helical threads configured to be screwed into a pedicle of a vertebra, or other bony region of a vertebra. In some embodiments, the shaft 18 may include a single thread, which may have a constant pitch or a variable pitch extending along the shaft 18. In other embodiments, the shaft 18 may include a plurality of threads extending helically around the shaft 18. For example, in some instances, the shaft 18 may include a first, distal region 40 having a first thread 36 forming a single threading, and a second, proximal region 42 including the first thread 36 and a second thread 38 forming a dual threading. The first thread 36 may include a start proximate the distal tip of the shaft 18 and may extend proximally along the distal region 40 and the proximal region 42 of the shaft 18. The second thread 38, which may include a start beginning at the transition between the distal region 40 and the proximal region 42, may extend proximally along the proximal region 42 and be located between adjacent windings of the first thread 36 throughout the proximal region 42. In other embodiments, the shaft 18 may have a different threaded configuration, if desired.

The housing 14 may include a base portion 24, from which the shaft portion 18 extends from, and first and second legs 26a, 26b extending from the base portion 24 on opposing sides of the housing 14. The first and second legs 26a, 26b may define an opening 28, which may be a threaded opening, extending into the housing 14 from an upper extent of the housing 14 opposite the base portion 24. Each of the first and second legs 26a, 26b may include a threaded portion for threadably engaging a threaded portion of a securing member 20, as described further herein. The housing 14 may additionally include a channel 30, such as a U-shaped channel, defined between the first and second legs 26a, 26b. The channel 30 may extend through the housing 14 from a first side 32 of the housing 14 to a second side 34 of the housing 14. The threaded opening 28 may intersect the channel 30. The channel 30 may be configured to receive a longitudinal connecting member of a vertebral stabilization system, such as a spinal rod, extending therethrough.

The bone screw 12 may include a securing member or closure member 20 configured to engage the housing 14 to secure a stabilizing member or connecting member (e.g., elongate rod or flexible cord) to the bone screw 12. The closure member 20 may be a threaded fastener, such as a set screw, having a threaded portion configured to threadably engage the threaded portions of the legs 26a, 26b of the housing 14. It is noted that in other embodiments, the legs 26a, 26b of the housing 14 may include external threaded portions configured to threadably engage a securing member 20 having an internal threaded portion. In such instances, the closure member 20 may be configured as a threaded nut, a threaded cap, or other internally threaded fastener, for example.

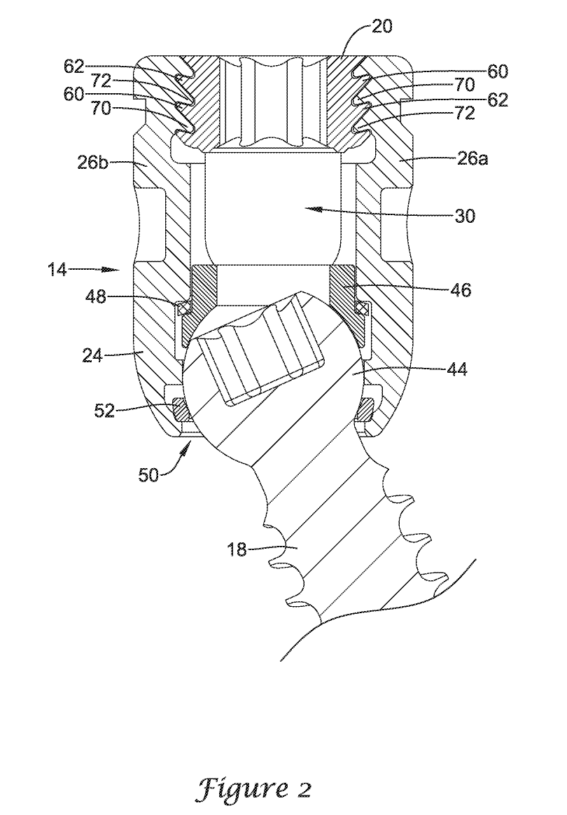

FIG. 2 is a cross-sectional view through the housing 14, further illustrating components of the bone screw 12. As shown in FIG. 2, in embodiments in which the bone screw 12 is a polyaxial screw, the base portion 24 may include a cavity, such as a spherical cavity, configured to pivotably receive the head 44, such as a spherically shaped head, of the shaft 18 therein. In some instances, the housing 14 may be configured such that the head 44 of the shaft 18 may be bottom loaded into the housing 14. In other words, the head 44 of the shaft 18 may be inserted into a cavity of the base portion 24 of the housing 14 through a lower opening 50 to assemble the shaft 18 to the housing 14. Once the head 44 is positioned in the cavity of the base portion 24, a retainer ring 52, such as a split ring, may be positioned in a groove formed in the base portion 24 to retain the head 44 in the cavity. In other embodiments, the housing 14 may be configured such that the head 44 of the shaft 18 is top loaded into the housing 14. In other words, the shaft 18 may be inserted through the housing 14 from the upper end such that the shaft 18 is passed out of the housing 14 through the lower opening 50 while the head 44 of the shaft 18 is retained in the cavity of the base portion 24 of the housing 14.

The bone screw 12 may also include an insert 46 positioned in the housing 14 configured to contact the head 44. In some instances, the insert 46 may include a spherically concave recess configured to receive a portion of the spherical head 44 therein. A biasing member, such as a wave washer 48, may be positioned in the housing 14 to exert a force against the insert 46 in order to press the insert 46 against the head 44 of the shaft 18. Thus, the insert 46 may frictionally engage the head 44, such that frictional forces between the insert 46 and the head 44 resist movement of the shaft 18 relative to the housing 14 until a sufficient force is applied to overcome the frictional forces.

As noted above, the closure member 20 have a threaded portion configured to threadably engage the threaded portions of the legs 26a, 26b of the housing 14. As shown in FIG. 2, the threaded portion of the closure member 20 may be a dual threaded portion, including a first thread 60 and a second thread 62 intertwined with the first thread 60. Correspondingly, the threaded portions of the legs 26a, 26b of the housing 14 may be dual threaded portions including a first thread 70 and a second thread 72 intertwined with the first thread 70.

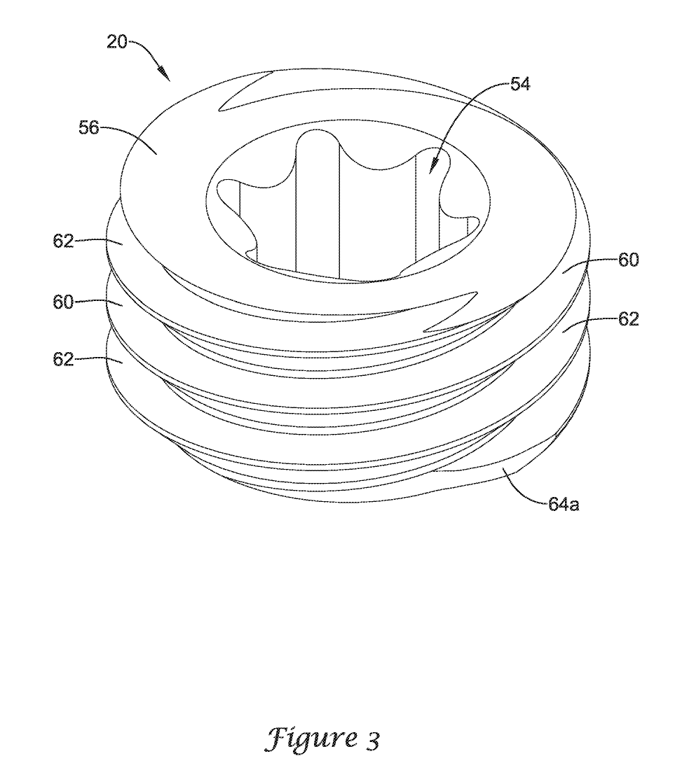

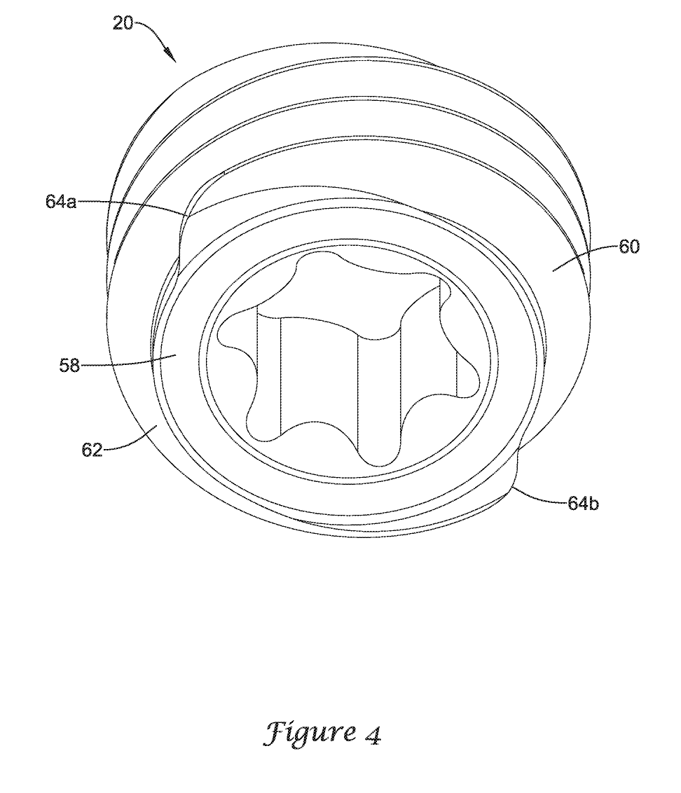

FIGS. 3 and 4 are perspective views further illustrating the closure member 20. The closure member 20 may include a driver engaging structure 54, such as an opening extending into or through the closure member 20 configured to receive and engage with a driver to rotate the closure member 20 into threaded engagement with the housing 14. The drive engaging structure 54 may have any desired configuration, such as a hex socket, a hexalobular socket (e.g., TORX® opening), or other desired engagement interface for receiving a driver to rotatably advance the closure member 20.

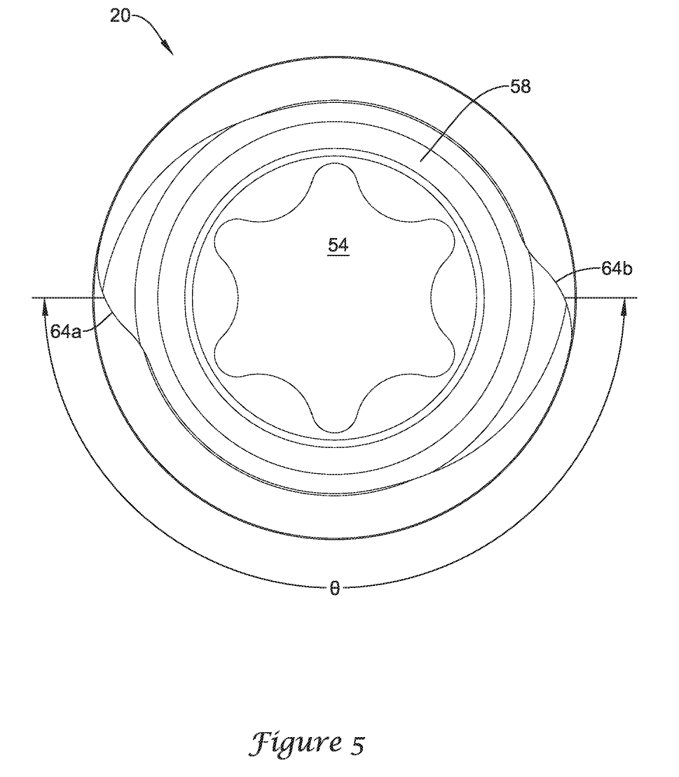

The first thread 60 may include a start 64a proximate the lower surface 58 of the closure member 20 and helically extend to the upper surface 56 of the closure member 20. Similarly, the second thread 62 may include a start 64b proximate the lower surface 58 of the closure member 20 and helically extend to the upper surface 56 of the closure member 20. The helical windings of the first thread 60 may alternate with helical windings of the second thread 62 from the lower surface 58 to the upper surface 56 of the closure member 20. As shown in FIG. 5, the start 64a of the first thread 60 may be oriented a desired angular displacement θ about the longitudinal axis of the closure member 20 from the start 64b of the second thread 62. For example, as shown in FIG. 5, the start 64a of the first thread 60 may be displaced 180° from the start 64b of the second thread 62 about the longitudinal axis of the closure member 20. However, in other embodiments, the angular displacement θ between the start 64a of the first thread 60 and the start 64b of the second thread 62 may be another angular amount, if desired.

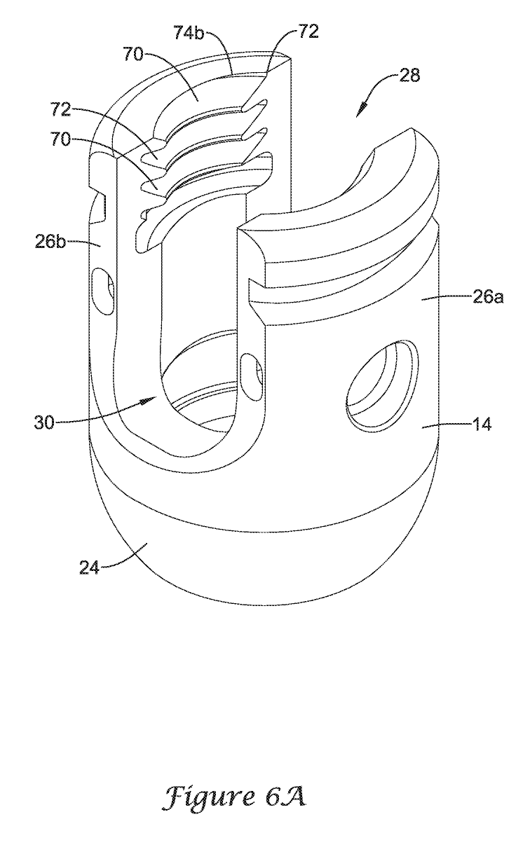

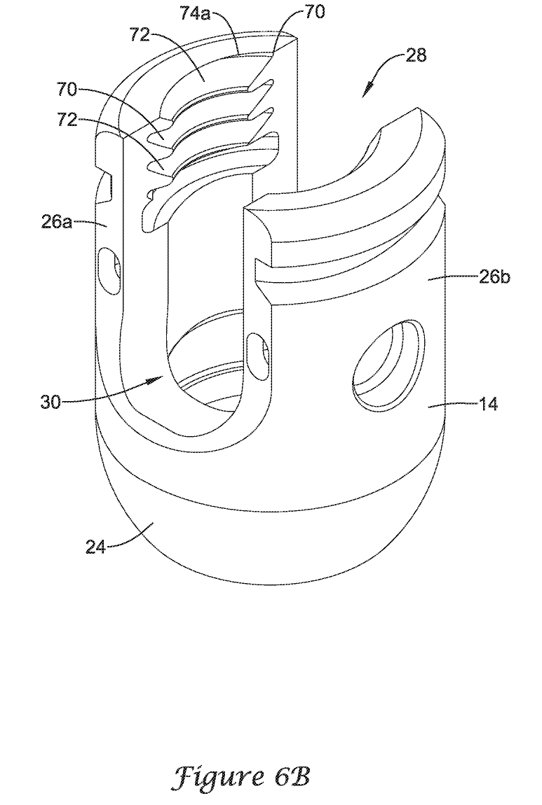

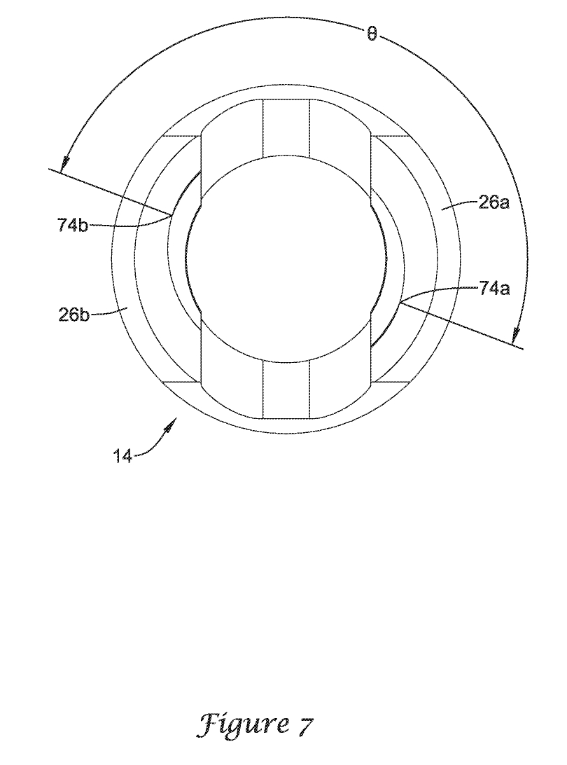

Turning to FIGS. 6A and 6B, the threaded portions of the first and second legs 26a, 26b of the housing 14 are further illustrated. The first thread 70 may include a start 74a proximate the upper extent of the first leg 26a of the housing 14 and helically extend along the opening 28. Similarly, the second thread 72 may include a start 74b proximate the upper extent of the second leg 26b and helically extend along the opening 28. The helical windings of the first thread 70 may alternate with helical windings of the second thread 72 along the opening 28 of the housing 14. In other words, each of the first and second legs 26a, 26b may including discontinuous portions of the first thread 70 alternating with discontinuous portions of the second thread 72 along an interior of the opening 28 of the housing 14. As shown in FIG. 7, the start 74a of the first thread 70 may be oriented a desired angular displacement θ about the longitudinal axis of the housing 14 from the start 74b of the second thread 72. For example, as shown in FIG. 7, the start 74a of the first thread 70 may be displaced 180° from the start 74b of the second thread 72 about the longitudinal axis of the housing 14. However, in other embodiments, the angular displacement θ between the start 74a of the first thread 70 and the start 74b of the second thread 72 may be another angular amount, if desired.

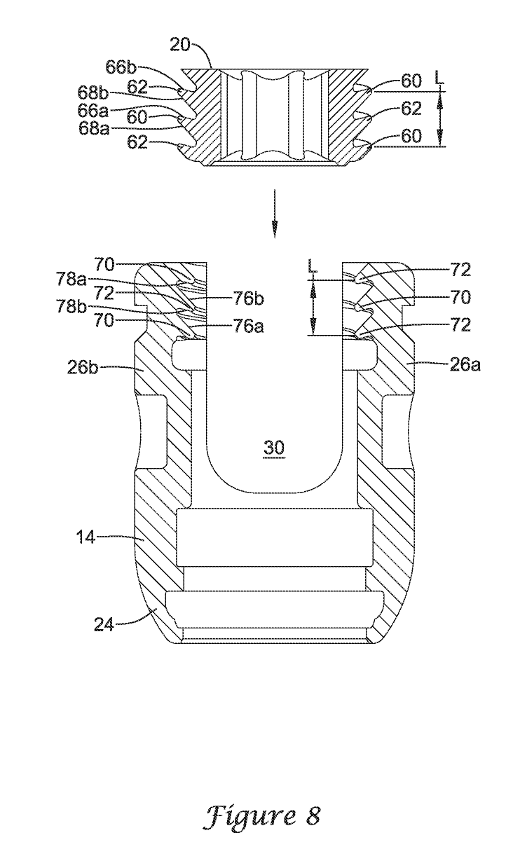

Referring to FIG. 8, it can be seen that the threaded portion of the closure member 20, and correspondingly, the threaded portions of the legs 26a, 26b of the housing 14 has a lead L (measured between crests of either the first thread 60, 70 or the second thread 62, 72) which is two times the pitch (measured between the crest of the first thread 60, 70 and the adjacent crest of the second thread 62, 72) of the threaded portion of the closure member 20 and the threaded portions of the legs 26a, 26b of the housing 14. Accordingly, when the closure member 20 is threadably engaged in the opening 28 of the housing 14, for every revolution (360°) of the closure member 20, the closure member 20 will be axially advanced into the housing 14 and against a connecting member positioned in the channel 30 twice as much as a closure member having a single thread having a lead equal to the pitch of the threading of the closure member.

When the closure member 20 is threadably engaged in the threaded opening 28 of the housing 14 between the first and second legs 26a, 26b, the upper flank 66a of the first thread 60 of the closure member 20 faces the lower flank 78b of the second thread 72 of the legs 26a, 26b, and the upper flank 66b of the second thread 62 of the closure member 20 faces the lower flank 78a of the first thread 70 of the legs 26a, 26b. Furthermore, the lower flank 68a of the first thread 60 of the closure member 20 faces the upper flank 76aof the first thread 70 of the legs 26a, 26b, and the lower flank 68b of the second thread 62 of the closure member 20 faces the upper flank 66b of the second thread 72 of the legs 26a, 26b. The threads 60, 62 of the exemplary closure member 20 shown in FIG. 8, and correspondingly the threads 70, 72 of the housing 14, have a reverse flank angle, meaning that both the upper flanks 66a, 66b and lower flanks 68a, 68b angle in the same direction from the central longitudinal axis. However, in other embodiments, the dual threads 60, 62 can have another threadform, if desired.

In order to secure a connecting member (e.g., elongate rod, elongate cord, spool, spindle, etc.) within the channel 30 of the housing 14, the closure member 20 may be threadably engaged in the threaded opening 28 between the legs 26a, 26b of the housing 20. The closure member 20 may be configured such that the start 64a of the first thread 60 of the closure member 20 threadably initiates threaded engagement with the first leg 26a of the housing 14 substantially simultaneously as the start 64b of the second thread 62 of the closure member 20 initiates threaded engagement with the second leg 26b of the housing 14. Thus, each of the first and second legs 26a, 26b of the housing 14 may be threadably engaged with the closure member 20 substantially simultaneously, or stated differently, the closure member 20 may initiate threaded engagement with each of the first and second legs 26a, 26b of the housing 14 substantially simultaneously. In other embodiments, the threads 60, 62 of the closure member 20 may be timed with the threads 70, 72 of the threaded opening 28 of the housing 14 such that the start 64a of the first thread 60 initiates threaded engagement with the first leg 26a of the housing 14 prior to the start 64b of the second thread 62 initiating threaded engagement with the second leg 26b of the housing 14, but before the start 64a of the first thread 60 initiates threaded engagement with the second leg 26b.

Furthermore, it can be seen that the closure member 20 will travel axially in the threaded opening 28 of the housing 14 twice as far per revolution (360°) as a conventional closure member having a single thread with the same thread pitch.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims.

1. (canceled)

2. A vertebral fastener comprising:
a housing and a bone engaging shaft extending from the housing, the housing including a first leg and a second leg extending from a base portion of the housing and defining a channel between the first leg and the second leg for receiving a connecting member therethrough, the bone engaging shaft having a distal region and a proximal region, wherein a first helical thread extends across the distal region and the proximal region, wherein a second helical thread extends across the proximal region and is located between adjacent windings of the first thread, and
a closure member configured to threadably engage the firs and second legs of the housing;
wherein the closure member includes a first thread and a second thread intertwined with the first thread, the first thread having a start proximate a lower surface of the closure member and the second thread having a start proximate the lower surface of the closure member.

3. The vertebral fastener of claim 2, wherein the start of the first thread of the closure member is angularly displaced about 180 degrees about a longitudinal axis of the closure member from the start of the second thread of the closure member.

4. The vertebral fastener of claim 2, wherein the closure member is configured such that the start of the first thread of the closure member threadably initiates threaded engagement with the first leg of the housing substantially simultaneously as the start of the second thread initiates threaded engagement with the second leg of the housing.

5. The vertebral fastener of claim 4, wherein the housing includes a first discontinuous thread and a second discontinuous thread intertwined with the first discontinuous thread.

6. The vertebral fastener of claim 5, wherein the first discontinuous thread of the housing has a start at an upper extent of the first leg of the housing and the second discontinuous thread of the housing has a start at an upper extent of the second leg of the housing.

7. The vertebral fastener of claim 6, wherein when the closure member is threadably engaged with the housing between the first and second legs, an upper flank of the first thread of the closure member faces a lower flank of the second thread of the housing, and an upper flank of the second thread of the closure member faces a lower flank of the first thread of the housing.

8. The vertebral fastener of claim 7, wherein when the closure member is threadably engaged with the housing between the first and second legs, a lower flank of the first thread of the closure member faces an upper flank of the first thread of the housing, and a lower flank of the second thread of the closure member faces an upper flank of the second thread of the housing.

9. A bone screw comprising:
a housing including a base portion and first and second legs extending from the base portion, the first and second legs defining a threaded opening extending into the housing from an upper extent of the housing, and a channel intersecting the threaded opening for receiving a connecting member therethrough;
a threaded shaft extending from the housing for threadably engaging a bony structure, the threaded shaft having a distal region and a proximal region, wherein a first helical thread extends across the distal region and the proximal region, wherein a second helical thread extends across the proximal region and is located between adjacent windings of the first thread; and
a threaded closure member configured to be threaded into the threaded opening of the housing between the first and second legs to secure a connecting member in the channel;
wherein the threaded closure member includes a first thread and a second thread intertwined with the first thread; and
wherein the threaded opening includes a first discontinuous thread and a second discontinuous thread intertwined with the first discontinuous thread.

10. The bone screw of claim 9, wherein adjacent windings of the first thread of the closure member alternate with adjacent windings of the second thread of the closure member.

11. The bone screw of claim 10, wherein the first discontinuous thread of the threaded opening alternates with the second discontinuous thread of the threaded opening along an inner surface of each of the first and second legs.

12. The bone screw of claim 11, wherein the first thread of the threaded closure member has a start proximate a lower surface of the closure member and the second thread of the threaded closure member has a start proximate the lower surface of the closure member.

13. The bone screw of claim 12, wherein the start of the first thread of the closure member is angularly displaced about 180 degrees about a longitudinal axis of the closure member from the start of the second thread of the closure member.

14. The bone screw of claim 12, wherein the closure member is configured such that the start of the first thread of the closure member threadably initiates threaded engagement with the first leg of the housing substantially simultaneously as the start of the second thread initiates threaded engagement with the second leg of the housing.

15. The bone screw of claim 9, wherein the first discontinuous thread of the threaded opening has a start at an upper extent of the first leg of the housing and the second discontinuous thread of the threaded opening has a start at an upper extent of the second leg of the housing.

16. The bone screw of claim 9, wherein when the closure member is threadably engaged in the threaded opening of the housing between the first and second legs, an upper flank of the first thread of the closure member faces a lower flank of the second discontinuous thread of the threaded opening, and an upper flank of the second thread of the closure member faces a lower flank of the first discontinuous thread of the threaded opening.

17. The bone screw of claim 9, wherein when the closure member is threadably engaged in the threaded opening of the housing between the first and second legs, a lower flank of the first thread of the closure member faces an upper flank of the first discontinuous thread of the threaded opening, and a lower flank of the second thread of the closure member faces an upper flank of the second discontinuous thread of the threaded opening.
